(19) United States
(12) Patent Application Publication
(10) Pub. No.: US 2025/0195136 A1
(43) Pub. Date: Jun. 19, 2025

(54) CARTRIDGE-BASED SINGLE-USE RF MEDICAL DEVICE WITH EXPANSION CARTRIDGE

(71) Applicant: Boston Scientific Scimed, Inc., Maple Grove, MN (US)

(72) Inventors: Steven Kinio, Pickering (CA); Christian Balkovec, Cambridge (CA)

(21) Appl. No.: 18/972,694

(22) Filed: Dec. 6, 2024

Related U.S. Application Data
(60) Provisional application No. 63/610,809, filed on Dec. 15, 2023.

(51) Int. Cl. A61B 18/14; A61B 17/00; A61B 18/00; A61B 34/10

(52) U.S. Cl. CPC A61B 18/1492; A61B 34/10; A61B 2017/0023; A61B 2017/00247; A61B 2017/00482; A61B 2018/00178; A61B 2018/00357; A61B 2018/00577

(57) ABSTRACT

A reusable connector for use with a single-use RF device for delivering therapy to a patient includes a housing having a proximal end and an opposite distal end. The proximal end is configured for connection to a control system for controlling delivery of energy to the single-use RF device. The distal end is configured for connection to the single-use RF device. A first slot is configured for receiving a first removable memory. The first removable memory is configured for authentication of a treatment plan to be performed by the single-use RF device. A second slot is configured for receiving a second removable memory. The second removable memory is configured for modification of the treatment plan. The first slot has a first shape and the second slot has a second shape different from the first shape.

### CROSS REFERENCE TO RELATED APPLICATIONS

This application claims priority to U.S. Provisional Patent Application No. 63/610,809 entitled “CARTRIDGE-BASED SINGLE-USE RF MEDICAL DEVICE WITH EXPANSION CARTRIDGE,” filed Dec. 15, 2023, which is herewith incorporated by reference in its entirety.

### TECHNICAL FIELD

The present disclosure relates to medical systems and methods for providing treatment to a tissue. More specifically, the present disclosure relates to reusable connectors with integral memory for use with radiofrequency (RF) devices.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels.

Additionally, RF devices can be used to penetrate tissue or create channels between body cavities. The use of RF energy to cut or vaporize biological tissue is a widely used technique in surgical applications. A target tissue can be vaporized using a conductive material that delivers RF energy of a specific voltage, frequency, and current density to it. This can be used to ligate or cut tissue with a cautery pen in general electrosurgery or to access locations in the body or vasculature through non-invasive percutaneous surgery. During percutaneous transseptal access from the right to the left atrium in the heart, for example, it is common to apply RF energy to puncture the interatrial septum (IAS).

### SUMMARY

Example 1 is a reusable connector for use with a single-use RF device for delivering therapy to a patient. The connector includes a housing having a proximal end and an opposite distal end. The proximal end is configured for connection to a control system for controlling delivery of energy to the single-use RF device. The distal end is configured for connection to the single-use RF device. A first slot is configured for receiving a first removable memory. The first removable memory is configured for authentication of a treatment plan to be performed by the single-use RF device.

Example 2 is the connector of Example 1, further comprising a second slot configured for receiving a second removable memory, the second removable memory being configured for modification of the treatment plan to be performed by the single-use RF device.

Example 3 is the connector of any of Examples 1 or 2, wherein the treatment plan includes delivering energy to one or more electrodes of the single-use RF device.

Example 4 is the connector of Example 3, wherein delivering energy to one or more electrodes of the single-use RF device includes delivering energy to a distal tip electrode in order to perforate tissue during a transseptal crossing procedure.

Example 5 is the connector of Example 3, wherein delivering energy to one or more electrodes of the single-use RF device includes delivering energy to the one or more electrodes to ablate tissue.

Example 6 is the connector of any of Examples 1-5, wherein the first slot has a first shape.

Example 7 is the connector of Example 6, wherein the second slot has a second shape different from the first shape.

Example 8 is the connector of Example 2, wherein the first removable memory and the second removable memory include eeprom, flash, SD, MMC, XD, or SDHC.

Example 9 is the connector of any of Examples 2-8, wherein the modification of the treatment plan includes changing a sequence of RF energy delivery.

Example 10 is the connector of any of Examples 2-8, wherein the modification of the treatment plan includes receiving control signals from an additional control system.

Example 11 is the connector of any of Examples 1-10, wherein the proximal end configured for connection to a control system includes a communication bus.

Example 12 is the connector of any of Examples 1-10, wherein the proximal end configured for connection to a control system includes a terminal pin.

Example 13 is the connector of any of Examples 1-10, wherein the proximal end configured for connection to a control system or the distal end configured for connection to the single-use RF device includes a male connector or a female connector.

Example 14 is the connector of any of Examples 1-13, the distal end configured for connection to the single-use RF device includes an over the wire connector.

Example 15 is the connector of any of Examples 1-14, further comprising one or more leads extending from the proximal end of the housing to the distal end of the housing.

Example 16 is a reusable connector for use with a single-use RF device for delivering therapy to a patient. The connector includes a housing having a proximal end and an opposite distal end. The proximal end is configured for connection to a control system for controlling delivery of energy to the single-use RF device. The distal end is configured for connection to the single-use RF device. A first slot is configured for receiving a first removable memory. The first removable memory is configured for authentication of a treatment plan to be performed by the single-use RF device. A second slot is configured for receiving a second removable memory. The second removable memory is configured for modification of the treatment plan. The first slot has a first shape and the second slot has a second shape different from the first shape.

Example 17 is the connector of Example 16, wherein the treatment plan includes delivering energy to one or more electrodes of the single-use RF device.

Example 18 is the connector of Example 17, wherein delivering energy to one or more electrodes of the single-use RF device includes delivering energy to a distal tip electrode in order to perforate tissue during a transseptal crossing procedure.

Example 19 is the connector of Example 17, wherein delivering energy to one or more electrodes of the single-use RF device includes delivering energy to the one or more electrodes to ablate tissue.

Example 20 is the connector of Example 16, wherein the first slot has a first shape.

Example 21 is the connector of Example 20, wherein the second slot has a second shape different from the first shape.

Example 22 is the connector of Example 16, wherein the first removable memory and the second removable memory include eeprom, flash, SD, MMC, XD, or SDHC.

Example 23 is the connector of Example 16, wherein the modification of the treatment plan includes changing a sequence of RF energy delivery.

Example 24 is the connector of Example 16, wherein the modification of the treatment plan includes receiving control signals from an additional control system.

Example 25 is the connector of Example 16, wherein the proximal end configured for connection to a control system includes a communication bus.

Example 26 is the connector of Example 16, wherein the proximal end configured for connection to a control system includes a terminal pin.

Example 27 is the connector of Example 16, wherein the proximal end configured for connection to a control system or the distal end configured for connection to the single-use RF device includes a male connector or a female connector.

Example 28 is the connector of Example 16, wherein the distal end configured for connection to the single-use RF device includes an over the wire connector.

Example 29 is the connector of Example 16, further comprising one or more leads extending from the proximal end of the housing to the distal end of the housing.

Example 30 is a system for applying energy to a patient. The system includes a single-use RF device having one or more electrodes and a reusable connector.

The connector includes a housing having a proximal end and an opposite distal end. The proximal end is configured for connection to a control system for controlling delivery of energy to the single-use RF device, and the distal end is configured for connection to the single-use RF device. A first slot is configured for receiving a first removable memory, the first removable memory being configured for authentication of a treatment plan to be performed by the single-use RF device. A second slot is configured for receiving a second removable memory, the second removable memory being configured for modification of the treatment plan.

Example 31 is the system of Example 30, wherein the treatment plan includes delivering energy to a distal tip electrode of the single-use RF device in order to perforate tissue during a transseptal crossing procedure.

Example 32 is the system of Example 30, wherein the treatment plan includes delivering energy to the one or more electrodes to ablate tissue.

Example 33 is a method for providing therapy to a patient using a single-use RF device. The method includes providing a single-use RF device having one or more electrodes. The method includes joining the single-use RF device to a connector, wherein the connector includes a first slot configured for receiving a first removable memory, the first removable memory being configured for authentication of a treatment plan. The method includes authenticating the treatment plan and delivering energy to the patient using the one or more electrodes.

Example 34 is the method of Example 33, wherein delivering energy to the patient using the one or more electrodes includes delivering energy to a distal tip electrode in order to perforate tissue during a transseptal crossing procedure.

Example 35 is the method of Example 33, wherein delivering energy to the patient using the one or more electrodes includes delivering energy to the one or more electrodes to ablate tissue.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 is schematic representation of a cartridge type connector for a single-use RF device, in accordance with an embodiment of the disclosure.

FIG. 3 is a perspective view of a cartridge type connector for a single-use RF device, in accordance with an embodiment of the disclosure.

FIGS. 4A-4E represent various shapes for custom shape inserts for use with a cartridge type connector, in accordance with an embodiment of the disclosure.

FIG. 5 is a flowchart illustration a method for providing therapy to a patient using a single-use RF device, in accordance with an embodiment of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

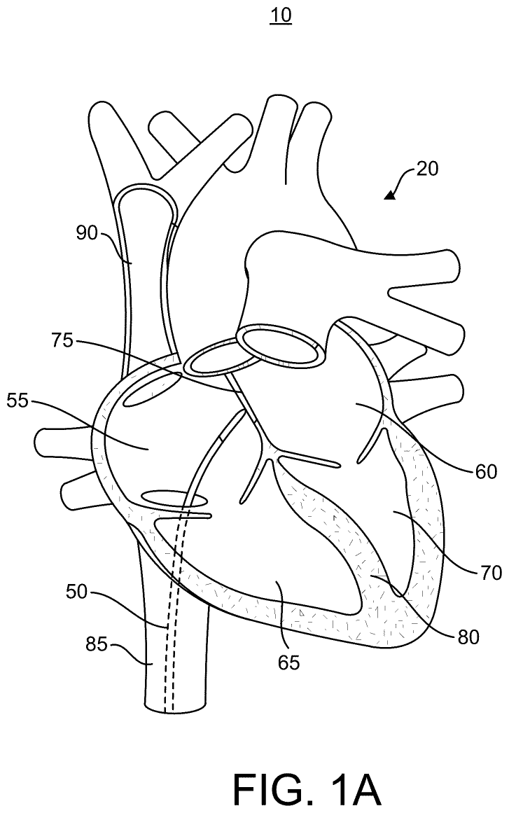

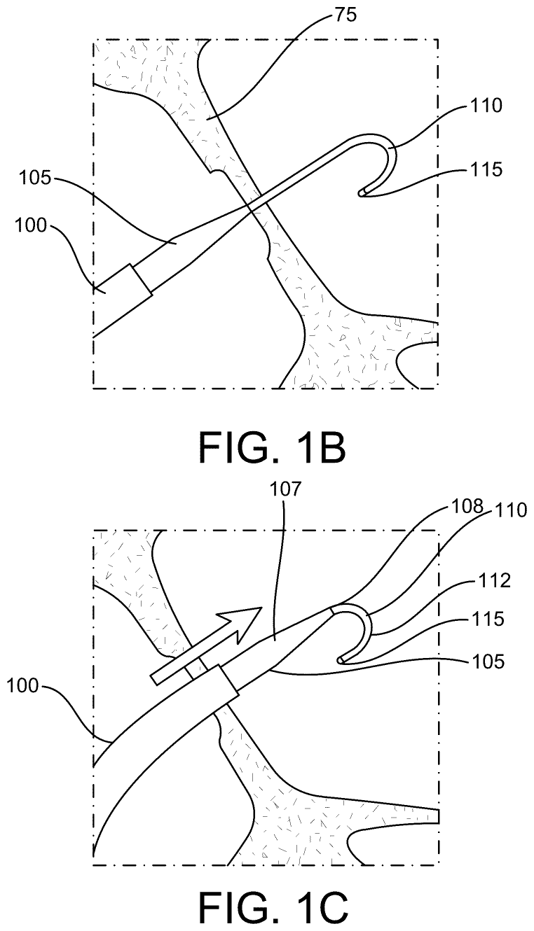

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. Additionally, in some embodiments, the RF perforation device 110 can be used as a guidewire. In this case, the RF perforation device 110 would be retracted into the dilator 105. Subsequently, the sheath 100, dilator 105, and the RF perforation device 110 would be retracted and positioned in the right atrium 55.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation apparatus 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s. In some RF puncture applications, a target delivery of at least 200 VRMS, for example 270 VRMS, is desired. In some applications, the puncture is typically accomplished with about 50W of power, and can take less than 1 second to achieve.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In some aspects, it may be desirable for the dilator 105, sheath 100, or RF perforation device 110 to include one or more surface electrode. The one or more surface electrode may be located on a distal portion of the dilator 105, sheath 100, or RF perforation device 110 for use in ablation, mapping, pacing, or sensing a parameter within a portion of the heart 20. The one or more surface electrode may be connected to an electroanatomical mapping (EAM) system, energy generator, or other diagnostic system.

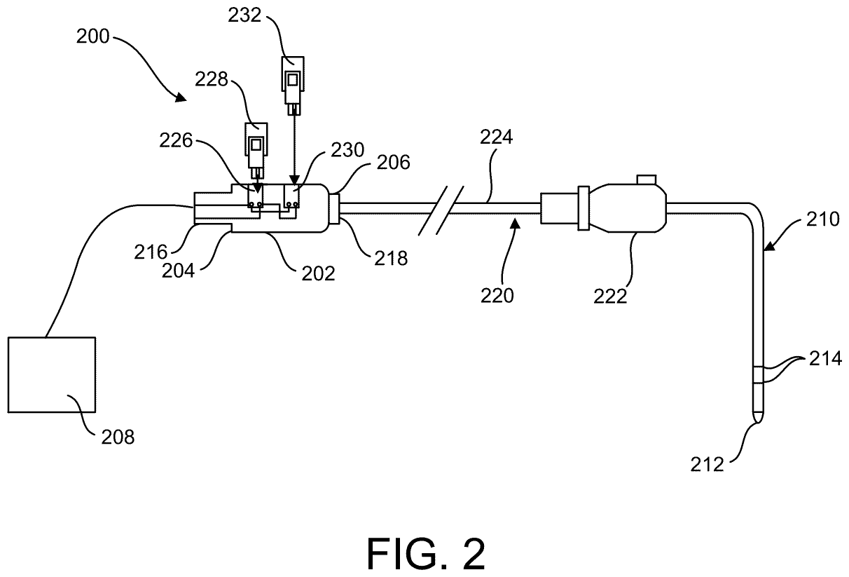

FIG. 2 is schematic representation of a cartridge type connector 200 for use with a single-use RF device 210, in accordance with an embodiment of the disclosure. The cartridge type connector 200 is configured to electrically couple a control system 208, for example an RF energy generator, to a single-use RF device 210. In addition to providing an electrical connection between a control system 208 and the single-use RF device 210, the cartridge type connector 200 allows for authenticating and modifying a treatment plan delivered to tissue by the single-use RF device 210.

The cartridge type connector 200 includes a housing 202 having a proximal end 204 and an opposite distal end 206. The proximal end 204 is configured for connection to a control system 208 for controlling delivery of energy to the single-use RF device 210. The distal end 206 is configured for connection to the single-use RF device 210. The single-use RF device 210 includes a plurality of electrodes, including a distal electrode 212, and a plurality of surface electrodes 214. In some embodiments, the single-use RF device 210 includes a single electrode.

The proximal end 204 includes one or more connectors 216 configured for coupling with the control system 208. In one embodiment, the one or more connectors 216 can include a communication bus. The communication bus includes wires or electronic pathways that enable transferring data, commands, and control signals between the control system 208 and the single-use RF device 210. The one or more connectors 216 can include other types of connectors, such as coaxial, pin connectors, or any male or female type connector.

The distal end 206 of the housing 202 includes one or more connectors 218 configured for removable coupling with the single-use RF device 210. The one or more connectors 218 can include various types of connectors such as coaxial, pin connectors, or any male or female type connector. In one embodiment, the one or more connectors 218 includes an over-the-wire (OTW) connector 220. The OTW connector 220 is configured to electrically connect with the single-use RF device 210 by receiving a portion of the single-use RF device 210 in a lumen or channel having one or more electrical contacts. The OTW connector 220 includes a coupling portion 222 and a cable 224 which allows for flexibility and movement between the housing 202 and the coupling portion 222.

In order to transmit signals from the proximal end 204 of the housing 202 to the distal end 206, one or more electrically conductive leads (not shown) extend from the proximal end 204 to the distal end 206. The one or more leads can take the form of a wire, an electrical trace or other conductor.

The housing 202 includes a first slot 226 configured for receiving a first removable memory 228. The first removable memory can include eeprom, flash, SD, MMC, XD, or SDHC, and other removable data storage devices. The first removable memory 228 is configured for authenticating a treatment plan to be performed by the single-use RF device 210. The first removable memory 228 includes information such as one or more keys or codes to allow for authenticating of a treatment plan. The control system 208 communicates with the first removable memory 228 to verify that a specific treatment plan is to be performed.

The information associated with the first removable memory 228 can also include specific instructions for a desired treatment plan. The instructions may dictate specific voltages, frequencies, current densities, and durations to be delivered by the distal electrode 212 or one or more of the plurality of surface electrodes 214 located on the RF device 210. In one aspect, a treatment plan includes delivering energy to one or more electrodes of the single-use RF device 210. In one aspect, delivering energy to one or more electrodes of the single-use RF device 210 includes delivering energy to a distal tip electrode 212 in order to perforate tissue during a transseptal crossing procedure. In another aspect, delivering energy to one or more electrodes of the single-use RF device 210 includes delivering energy to the one or more surface electrodes 214 or the distal tip electrode 212 to ablate tissue.

A second slot 230 is located on the housing 202 and is configured for receiving a second removable memory 232. The second removable memory 232 can include eeprom, flash, SD, MMC, XD, or SDHC, and other removable data storage devices. The second removable memory 232 is configured for modification of the treatment plan to be performed by the single-use RF device 210. The second removable memory 232 includes information, that when communicated to the control system 208, can change or modify the treatment plan. Modification of the treatment plan includes changing a sequence, duration, voltage, or frequency of RF energy delivery. In some aspects, the information associated with the second removable memory 232 may be communicated to an additional control system. Modification of the treatment plan can include receiving control signals from an additional control system.

The first slot 226 and the second slot 230 can be configured for receiving the same type of removable memory. Alternatively, the first slot 226 and the second slot 230 can be configured for receiving different types of removable memory. In some aspects, the first slot 226 has a first shape and the second slot 230 has a second shape different from the first shape. The first slot 226 and the second slot 230 may also include a means for providing tactile feedback to a user upon insertion of the removable memory. The housing 220 can include a movable cover to selectively cover the first slot 226, the second slot 230, or both the first slot 226 and the second 230.

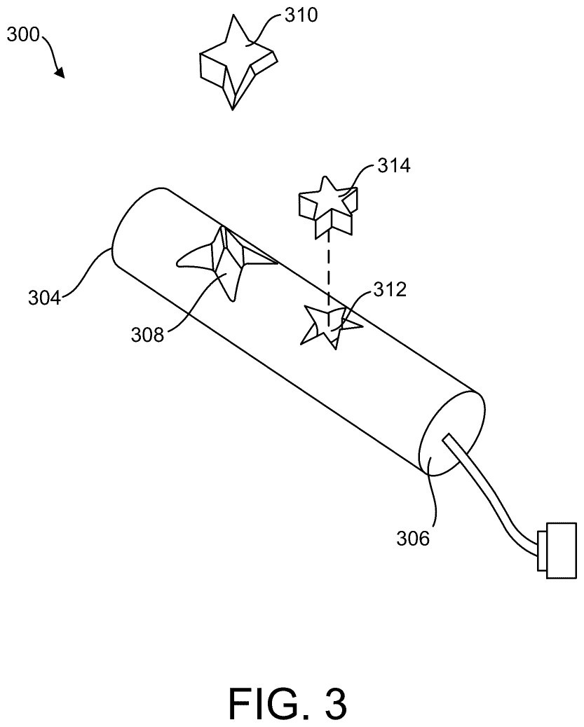

FIG. 3 is a perspective view of a cartridge type connector 300 for a single-use RF device (not shown), in accordance with an embodiment of the disclosure. The cartridge type connector 300 in FIG. 3 includes a housing 302 having a proximal end 304 and an opposite distal end 306. The proximal end 304 is configured for connection to a control system for controlling delivery of energy to the single-use RF device, and can include any type of connector such as those discussed above. The distal end 306 is configured for connection to the single-use RF device, and can include any type of connector such as those discussed above.

The housing 302 includes a first slot 308 having a custom shape, for example a cross shape, configured for receiving a first removable memory 310. The first removable memory 310 is configured for authentication of a treatment plan to be performed by the single-use RF device. The first removable memory 310 can include eeprom, flash, SD, MMC, XD, or SDHC, and other removable data storage devices and has a shape configured to mate with the first slot 308.

The housing 302 includes a second slot 312 having a custom shape, for example a star shape, configured for receiving a second removable memory 314. The second removable memory is configured for modification of the treatment plan to be performed by the single-use RF device. The second removable memory 314 can include eeprom, flash, SD, MMC, XD, or SDHC, and other removable data storage devices and has a shape configured to mate with the second slot 312.

As illustrated, the first slot 308 has a first shape and the second slot 312 has a second shape different from the first shape. This allows for easily differentiating between the first removable memory 310 for carrying out a first function, and the second removable memory 314 that carries out a second function. In one aspect, the first slot 308 has a first shape and the second slot 312 has a second shape that is identical to the first shape.

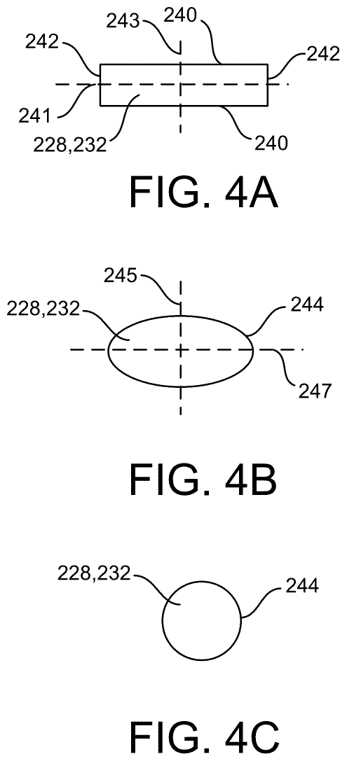

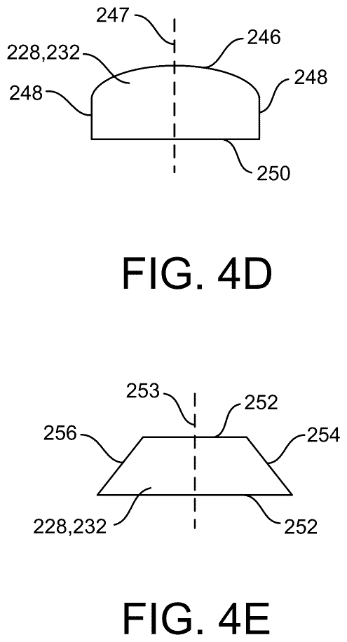

FIGS. 4A-4E represent various shapes for custom shape inserts or removable memory for use with a cartridge type connector, in accordance with an embodiment of the disclosure. FIG. 4A illustrates a removable memory 228, 232 having a rectangular cross-section. The rectangular cross-section includes a first pair of surfaces 240 that are orthogonal to a second pair of surfaces 242. The rectangular cross-section is symmetric over a first line of symmetry 241 and a second line of symmetry 243. FIG. 4B illustrates removable memory 228, 232 having an oval cross-section. The oval cross-section includes a single surface 244. The oval cross-section is symmetric over a first line of symmetry 245 and a second line of symmetry 247 FIG. 4C illustrates a removable memory 228, 232 having a circular cross-section. Like the oval cross-section, the circular cross-section includes a single surface 244 but is symmetric over an infinite number of lines of symmetry (not shown). FIG. 4D illustrates a removable memory 228, 232 having a dome shaped cross-section. The dome shaped cross-section includes a curved surface 246, and a first pair of parallel surfaces 248 that are orthogonal to a flat surface 250 opposite of the curved surface 246. The dome shape cross-section is symmetric over only one line of symmetry 247. FIG. 4E illustrates a removable memory 228, 232 having a polygonal cross-section. The polygonal cross-section includes a pair of parallel surfaces 252 that are intersected by a first angled surface 254 and a second angled surface 256. The polygonal cross-section is symmetric over a single line of symmetry 253. While each cross-section illustrated in FIGS. 4A-4E are symmetric over at least one line of symmetry, non-symmetric cross-sections are also contemplated for use as custom shaped inserts or removable memory.

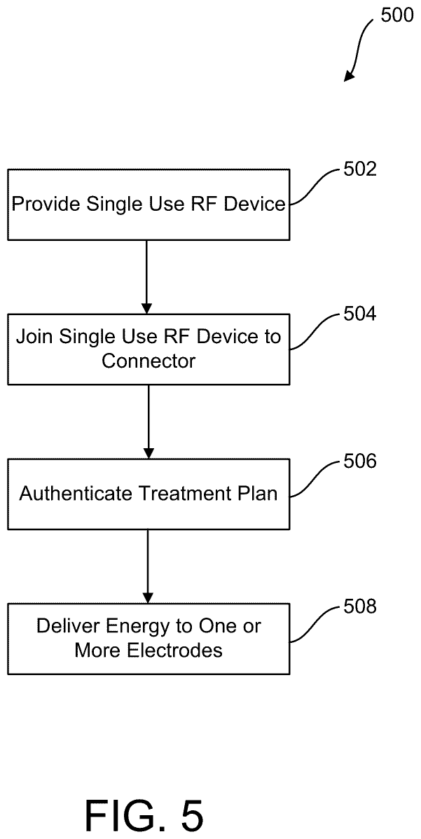

FIG. 5 is a flowchart illustrating a method 500 for providing therapy to a patient using a single-use RF device, in accordance with an embodiment of the disclosure. Step 502 of the method includes providing a single-use RF device having one or more electrodes. Step 504 includes joining the single-use RF device to a connector. The connector includes a first slot configured for receiving a first removable memory, the first removable memory being configured for authentication of a treatment plan. Step 506 includes authenticating the treatment plan. Following authentication, step 508 includes delivering energy to the patient using the one or more electrodes.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A reusable connector for use with a single-use RF device for delivering therapy to a patient, the connector comprising:
a housing having a proximal end and an opposite distal end, the proximal end being configured for connection to a control system for controlling delivery of energy to the single-use RF device, and the distal end being configured for connection to the single-use RF device;
a first slot configured for receiving a first removable memory, the first removable memory being configured for authentication of a treatment plan to be performed by the single-use RF device; and
a second slot configured for receiving a second removable memory, the second removable memory being configured for modification of the treatment plan;
wherein the first slot has a first shape and the second slot has a second shape different from the first shape.

2. The connector of claim 1, wherein the treatment plan includes delivering energy to one or more electrodes of the single-use RF device.

3. The connector of claim 2, wherein delivering energy to one or more electrodes of the single-use RF device includes delivering energy to a distal tip electrode in order to perforate tissue during a transseptal crossing procedure.

4. The connector of claim 2, wherein delivering energy to one or more electrodes of the single-use RF device includes delivering energy to the one or more electrodes to ablate tissue.

5. The connector of claim 1, wherein the first slot has a first shape.

6. The connector of claim 5, wherein the second slot has a second shape different from the first shape.

7. The connector of claim 1, wherein the first removable memory and the second removable memory include eeprom, flash, SD, MMC, XD, or SDHC.

8. The connector of claim 1, wherein the modification of the treatment plan includes changing a sequence of RF energy delivery.

9. The connector of claim 1, wherein the modification of the treatment plan includes receiving control signals from an additional control system.

10. The connector of claim 1, wherein the proximal end configured for connection to a control system includes a communication bus.

11. The connector of claim 1, wherein the proximal end configured for connection to a control system includes a terminal pin.

12. The connector of claim 1, wherein the proximal end configured for connection to a control system or the distal end configured for connection to the single-use RF device includes a male connector or a female connector.

13. The connector of claim 1, wherein the distal end configured for connection to the single-use RF device includes an over the wire connector.

14. The connector of claim 1, further comprising one or more leads extending from the proximal end of the housing to the distal end of the housing.

15. A system for applying energy to a patient, the system comprising:
a single-use RF device having one or more electrodes; and
a reusable connector, the connector comprising:
a housing having a proximal end and an opposite distal end, the proximal end being configured for connection to a control system for controlling delivery of energy to the single-use RF device, and the distal end being configured for connection to the single-use RF device;
a first slot configured for receiving a first removable memory, the first removable memory being configured for authentication of a treatment plan to be performed by the single-use RF device; and
a second slot configured for receiving a second removable memory, the second removable memory being configured for modification of the treatment plan.

16. The system of claim 15, wherein the treatment plan includes delivering energy to a distal tip electrode of the single-use RF device in order to perforate tissue during a transseptal crossing procedure.

17. The system of claim 15, wherein the treatment plan includes delivering energy to the one or more electrodes to ablate tissue.

18. A method for providing therapy to a patient using a single-use RF device, the method comprising:
providing a single-use RF device having one or more electrodes;
joining the single-use RF device to a connector, wherein the connector includes a first slot configured for receiving a first removable memory, the first removable memory being configured for authentication of a treatment plan;
authenticating the treatment plan; and
delivering energy to the patient using the one or more electrodes.

19. The method of claim 18, wherein delivering energy to the patient using the one or more electrodes includes delivering energy to a distal tip electrode in order to perforate tissue during a transseptal crossing procedure.

20. The method of claim 18, wherein delivering energy to the patient using the one or more electrodes includes delivering energy to the one or more electrodes to ablate tissue.
